(19) United States
(12) Patent Application Publication
(10) Pub. No.: US 2025/0221991 A1
(43) Pub. Date: Jul. 10, 2025

(54) COMBINATION THERAPY FOR THE TREATMENT OF CANCER METASTASES

(71) Applicant: University of Cincinnati, Cincinnati, OH (US)

(72) Inventor: Georg F. Weber, Milford, OH (US)

(73) Assignee: University of Cincinnati, Cincinnati, OH (US)

(21) Appl. No.: 19/010,832

(22) Filed: Jan. 6, 2025

Related U.S. Application Data
(60) Provisional application No. 63/617,863, filed on Jan. 5, 2024.

(51) Int. Cl. A61K 31/506; A61K 31/10; A61K 31/122; A61K 31/16; A61K 31/196; A61K 33/24; A61P 35/04

(52) U.S. Cl. CPC A61K 31/506; A61K 31/10; A61K 31/122; A61K 31/16; A61K 31/196; A61K 33/24; A61P 35/04

(57) ABSTRACT

A method of treating cancer metastasis in a subject in need thereof is provided, the method including administering to the subject a combination therapy including: (a) a vascularization and/or tissue remodeling inhibitor; (b) a suppressor of oxidative metabolism; and (c) an ion homeostasis modulator. Pharmaceutical compositions directed to the combination of therapeutic agents are also provided.

### CROSS REFERENCE TO RELATED APPLICATION

This application claims priority to U.S. Provisional Application Ser. No. 63/617,863, filed Jan. 5, 2024, the entire contents of which are incorporated herein by reference.

### STATEMENT REGARDING FEDERALLY SPONSORED RESEARCH OR DEVELOPMENT

This invention was made with government support under CA224104 and TR001425 awarded by the National Institutes of Health. The government has certain rights in the invention.

### TECHNICAL FIELD

The present disclosure relates to the field of cancer therapy. Specifically, this disclosure relates to treatment of cancer metastases by administering a combination drug therapy.

### BACKGROUND

Although the chemotherapy of primary tumors has been increasingly successful, the drug treatment of metastases has been compromised by low success rates. A likely reason lies in the clinical proclivity to treat the disseminated growths with the same agents that have been devised to counteract the primary tumors. Metastases, however, evolve their gene expression patterns to differentiate from their originating tumors as well as from the implantation organ. Site-specific changes would be difficult to control with chemotherapy, as each site of dissemination would require a separate set of drugs.

A need exists for improved, effective therapies for the treatment of cancer metastases.

### SUMMARY

Embodiments of the present disclosure relate to methods and combination therapies for the treatment of cancer metastases. Combination therapy, targeted to the molecular mechanisms of metastasis, rather than treating the primary tumor or destination tissue, offers an innovative approach to treating metastatic cancers that have a traditionally poor prognosis and outcome. Accordingly, provided herein are combination therapies and compositions that target metastasis.

In one embodiment, a method of treating cancer metastasis in a subject in need thereof is provided, the method including administering to the subject a combination therapy comprising: a vascularization and/or tissue remodeling inhibitor; a suppressor of oxidative metabolism; and an ion homeostasis modulator.

In another embodiment, a pharmaceutical composition is provided, comprising: a vascularization and/or tissue remodeling inhibitor; a suppressor of oxidative metabolism; an ion homeostasis modulator; and at least one pharmaceutically-acceptable excipient.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1A graphically depicts the metastasis count in lung dissemination after intravenous injection with 4T1 cells and combination treatment, starting on Day 0.

FIG. 1B graphically depicts the metastasis count in lung dissemination after intravenous injection with 4T1 cells followed by delayed combination treatment, starting on Day 5.

FIG. 1C graphically depicts the metastasis count in lung dissemination after intravenous injection with B16-F10 cells and combination treatment, starting on Day 0.

FIG. 1D graphically depicts the tumor burden in peritoneal dissemination after subcutaneous injection with B16-F10 cells followed by combination treatment, starting on Day 0 and Day 5.

FIG. 1E graphically depicts the tumor burden in peritoneal dissemination after subcutaneous injection with B16-F10 cells and combination treatment, starting on Day 0.

FIG. 2A graphically depicts the effects of combination treatment on B16-F10 cell colony formation in soft agar.

FIG. 2B graphically depicts the effects of combination treatment on PANC-1 cell colony formation in soft agar.

FIG. 2C graphically depicts lung dissemination after intravenous injection of B16-F10 cells, with treatment starting on Day 0.

FIG. 2D graphically depicts the tumor burden in peritoneal dissemination after subcutaneous injection of B16-F10 cells, with treatment starting on Day 5.

FIG. 3 graphically depicts the effects of combination treatment on MDA-MB-231 cells, PANC-1 cells and Mia PaCa-2 cell colony formation in soft agar.

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

Embodiments of the present disclosure generally relate to methods and compositions for the treatment of cancer metastases in a subject in need thereof. Metastatic cancers are associated with poor prognosis. The presently disclosed methods for treating metastases are based on gene expression indicators of metastasis formation, rather than conventional treatments of the originating primary tumor(s). As described in greater detail herein, a combination therapy that targets tissue remodeling, oxidative metabolism, and ion homeostasis, and optionally silences extracellular matrix interactions functions to disrupt the mechanisms driving metastasis formation, providing a metastasis-specific approach to cancer therapy.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

As used herein, the term “subject” generally refers to a living being (e.g., animal or human) capable of suffering from cancer. In a specific embodiment, the subject is a mammal, such as a human, rat, mouse, monkey, horse, cow, pig, dog, cat, guinea pig, etc. In a more specific embodiment, the subject is a human subject, a rat, or a mouse. In a more specific embodiment, the subject is a human.

The terms “treat,” “treatment,” and “treating,” as used herein, refer to a method of alleviating or abrogating a disease, disorder, and/or symptoms thereof. In a specific embodiment, the disease or disorder is cancer metastasis. In a more specific embodiment, the cancer metastasis is a solid tumor. In another specific embodiment, the cancer metastasis originates from a primary cancer selected from the group consisting of pancreatic cancer, breast cancer, colorectal cancer, and rhabdomyosarcoma.

As used herein, the terms “administer” or “administration” may comprise administration routes such as enteral (e.g., oral, sublingual, buccal, or rectal), parenteral (e.g., intravenous, intramuscular, subcutaneous, intraarterial, intrathecal, intratumoral, etc.), intranasal, inhaled, vaginal, transdermal, etc., so long as the route of administration results in treatment of cancer, including cancer metastases. In specific embodiments, the administration route is enteral or parenteral. In another specific embodiment, the administration route is intravenous or intratumoral. In another specific embodiment, the administration route is oral. In another specific embodiment, the therapeutic agents comprising the combination therapy may be administered via different routes. For example, one or more agents of the combination therapy may be administered enterally, while other agent(s) may be administered parenterally. The skilled artisan will appreciate that each therapeutic agent included in a combination therapy as disclosed herein may have a preferred route of administration.

“Co-administered,” as used herein, refers to administration of the disclosed therapeutic agents, such that administered agents can simultaneously achieve a physiological effect, e.g., in a recipient subject. The therapeutic agents, however, need not be administered together. In certain embodiments, administration of one agent can precede administration of the other(s). Simultaneous physiological effect need not necessarily require presence of all agents in the circulation at the same time. However, in certain embodiments, co-administering typically results in the administered agents being simultaneously present in the subject. Thus, in embodiments, the therapeutic agents of a combination therapy may be administered concurrently or sequentially.

“Effective amount,” as used herein, refers to an amount of an agent sufficient to achieve a desired biological effect. Effective amounts will vary based on a subject's age, body weight, condition, and the like, and may be determined by one of skill in the art in view of the present disclosure. The compositions of the present disclosure can be administered by either single or multiple dosages of an effective amount. In embodiments, the effective amount of an agent is an amount sufficient to treat a cancer metastasis. In specific embodiments, the effective amount is an amount sufficient to prevent, stop, inhibit, or suppress the growth of a metastatic tumor. In another specific embodiment, the effective amount is an amount sufficient to reduce metastatic tumor mass.

“Subtherapeutic dose” refers to a dose of a therapeutic agent that is lower than the typical dose of the therapeutic agent when administered alone for the treatment of a disease, such as cancer or cancer metastases. In some aspects, a subtherapeutic dose may be 10%, 15%, 20%, 25%, 30%, 35%, 40%, 45%, 50%, 55%, 60%, 65%, 70%, 75%, 80%, 85%, or 90% of the therapeutic dose for that agent, when administered as a monotherapy.

The term “pharmaceutically-acceptable excipient,” as used herein, means any physiologically inert, pharmacologically inactive material known to one skilled in the art, which is compatible with the physical and chemical characteristics of the particular active agent(s) selected for use. Pharmaceutically acceptable excipients include, but are not limited to, polymers, resins, plasticizers, fillers, lubricants, diluents, binders, disintegrants, solvents, co-solvents, buffer systems, surfactants, preservatives, sweetening agents, flavoring agents, pharmaceutical grade dyes or pigments, and viscosity agents. In specific embodiments, the pharmaceutically-acceptable excipients are excipients suitable for intravenous formulations, intratumoral formulations, or oral formulations.

“Broad spectrum,” as used herein, refers to a therapeutic agent that exhibits efficacy across multiple targets. For example, and without being bound by theory, a broad spectrum inhibitor refers to a compound that inhibits multiple molecular targets, which may include receptors, enzymes, signaling proteins, or other biomolecules.

### Combination Therapy for Treating Metastases

Disseminated cancerous growths are generally characterized by a program of gene expression that induces the oxidative metabolism, activates vascularization/tissue remodeling, alters ion homeostasis, and silences extracellular matrix interactions. This signature distinguishes the metastases from their originating primary tumors as well as from their target host tissues. The core program of metastasis is common to, and unique for all solid tumor metastases, thus providing a promising target for metastasis-specific therapy.

The rationale for the presently disclosed methods and compositions is rooted in previous research that utilized the gene expression profiles from 653 Gene Expression Omnibus (GEO) datasets and a mouse model to investigate whether the signatures by diverse cancers in various metastatic sites display common features. There is evidence that the gene expression core program of metastasis is activated at the time of release from the primary growth and remains active in the foreign microenvironment of the target site, where continuous survival signals are required. The lasting activity of this genetically encoded core program makes it a suitable target for both the treatment of existing metastases and the prevention of new metastases.

As described herein, methods of the disclosure generally relate to a method of treating cancer metastases in a subject in need thereof by administering a combination therapy to the subject. Optionally, the cancer metastasis originates from a primary cancer selected from the group consisting of pancreatic cancer, breast cancer, colorectal cancer, or rhabdomyosarcoma. However, it should be appreciated that the core program is common to metastases of diverse cancers and is not limited to any particular primary cancer or subset of primary cancers.

In some embodiments, the combination therapy includes a plurality of therapeutic agents that target metastasis. Without being bound, the combination therapy may include three therapeutic agents, four therapeutic agents, five therapeutic agents, six therapeutic agents, seven therapeutic agents, eight therapeutic agents, nine therapeutic agents, ten therapeutic agents, etc. Optionally, the therapeutic agents include one or more of: a vascularization and/or tissue remodeling inhibitor, a suppressor of oxidative metabolism, an ion homeostasis modulator, and/or a silencer of extracellular matrix interactions. In some embodiments, the combination therapy includes at least one vascularization and/or tissue remodeling inhibitor, at least one suppressor of oxidative metabolism, and at least one ion homeostasis modulator.

Optionally, a therapeutic agent suppresses deadherent cell expansion at doses low enough to not affect adherent cells. It will be appreciated that a therapeutic agent that suppresses deadherent cell expansion operates by targeting the specific survival and proliferative pathways that enable metastatic cells to thrive despite their detachment from the extracellular matrix. This enables effective treatment of cancer metastases while limiting unwanted side effects.

A therapeutic agent may also inhibit the in vitro formation of soft agar colonies. Soft agar assays mimic the conditions encountered by cancer cells during dissemination and colonization of distant tissues. Inhibiting colony formation indicates the therapeutic agent's ability to impede anchorage-independent growth of cells. This disruption indicates impairment of the metastatic process, as anchorage-independent growth is closely linked to the ability of cancer cells to survive in suspension, evade anoikis (detachment-induced apoptosis), and establish new metastatic sites in distant tissues.

In some embodiments, the therapeutic agent demonstrates increased efficacy when used in combination. The increased efficacy observed in the working examples when therapeutic agents are used in combination arises from the ability to target multiple, complementary aspects of the metastatic process simultaneously. Each therapeutic agent within the combination therapy may inhibit distinct pathways or mechanisms for cancer cell survival, proliferation, and/or colonization.

In some embodiments, a therapeutic agent is a vascularization and/or tissue remodeling inhibitor. Optionally, the vascularization and/or tissue remodeling inhibitor is an inhibitor of angiogenesis. In some embodiments, the therapeutic agent inhibits one or more molecular targets or pathways. Optionally, the molecular target and/or pathway is involved in angiogenesis. In some embodiments, expression of the molecular target is associated with increased metastatic potential. Exemplary, non-limiting molecular targets include vascular endothelial growth factor ligands (e.g., VEGF-A, VEGF-B, VEGF-C, VEGF-D, PIGF, etc.), vascular endothelial growth factor receptors (e.g., VEGFR-1 (Flt-1), VEGFR-2 (KDR/Flk-1), VEGFR-3 (Flt-4)), matrix metalloproteinases (MMPs) (e.g., MMP1, MMP2, MMP3, MMP7, MMP8, MMP9, MMP10, MMP11, MMP12, MMP13, MMP20, MMP21, MMP26, MMP27, MMP28), vascular endothelial growth factor co-receptors (e.g., NRP1, NRP2), platelet-derived growth factor ligands (e.g., PDGF-AA, PDGF-BB, PDGF-CC, PDGF-DD), platelet-derived growth factor receptors (e.g., PDGFR-α, PDGFR-β), fibroblast growth factor ligands (e.g., FGF1, FGF2, FGF3, FGF4, FGF5, FGF6, FGF, etc.), fibroblast growth factor receptors (e.g., FGFR1, FGFR2, FGFR3, FGFR4), angiopoietins (e.g., Ang-1, Ang-2), tie receptors (e.g., Tie-1, Tie-2), integrins (e.g., αvβ3, αvβ5, α5β1), Notch signaling ligands (e.g., DLL4, Jagged1, Jagged2), Notch signaling receptors (e.g., Notch1, Notch2, Notch3, Notch4), inflammatory cytokines (e.g., IL-6, IL-8, TNF-α), combinations thereof, and the like.

In some embodiments, the therapeutic agent is a vascular endothelial growth factor receptor (VEGFR) inhibitor. In some embodiments, the vascularization inhibitor is a broad spectrum VEGFR inhibitor. Exemplary, non-limiting VEGFR inhibitors include pazopanib, bevacizumab, sunitinib, cabozantinib, sorafenib, regorafenib, lenvatinib, nintedanib, apatinib, axitinib, vandetanib, tivozanib, ramucirumab, fruquintinib, ponatinib, and combinations thereof. In some embodiments, the VEGFR inhibitor is pazopanib.

In situ, tumor-dependent ECM remodeling is involved in dissemination. In some embodiments, the therapeutic agent is a matrix metalloproteinase (MMP) inhibitor, which targets remodeling. In some embodiments, the tissue remodeling inhibitor is a broad spectrum MMP inhibitor. Exemplary, non-limiting MMP inhibitors include marimastat, ilomastat, batimastat, MMI-270, MMI-166, prinomastat, ABT-770, cipemastat, RS-130830, CAS Reg. No. 239796-97-5, rebimastat, tanomastat, Ro 28-2653, and combinations thereof. In some embodiments, the MMP inhibitor is marimastat.

In some embodiments, a therapeutic agent is a suppressor of oxidative metabolism. Deadherent survival and growth require induction of the oxidative metabolism. Without being bound by theory, this may be due to an increased need for ATP once contact to the substratum and to other cells is lost. Therefore, distinct from the Warburg effect in primary tumors, increased mitochondrial respiration is needed for cell survival during metastasis.

In some embodiments, the suppressor of oxidative metabolism is a hydroxyl radical scavenger or a peroxide scavenger. A hydroxyl radical scavenger is a molecule or substance that neutralizes hydroxyl radicals generated during oxidative stress. A peroxide scavenger is a molecule or substance that neutralizes hydrogen peroxide or other peroxides generated during oxidative stress. Exemplary, non-limiting hydroxyl radical or peroxide scavengers include dimethyl sulfoxide (DMSO), atovaquone, edaravone, taurine, N-acetylcysteine (NAC), glutathione, mannitol, curcumin, fullerenes, cerium oxide nanoparticles, combinations thereof, and the like. In embodiments, the suppressor of oxidative metabolism is DMSO or atovaquone.

In some embodiments, a therapeutic agent is an ion homeostasis modulator. Ion homeostasis is substantially altered in cancer cells and even more so in metastasis. Ion homeostasis modulators may impact the deadherent survival of cancer cells. Exemplary ions that may be targets for modulation encompass a variety of ions involved in cellular processes and physiological regulation. These ions include, but are not limited to sodium, potassium, chloride, copper, and calcium.

In some embodiments, the ion homeostasis modulator is a sodium-potassium-chloride cotransporter (NKCC) inhibitor, thereby reducing the transport of sodium, potassium, and chloride ions across cell membranes. Metastatic cancer cells often have altered ion gradients compared to normal cells, which enables them to survive in hostile environments, such as low-oxygen (hypoxic) regions, or during circulation through the bloodstream. The ability of NKCC to regulate cell volume and intracellular osmolarity is especially important for metastatic cells as they navigate the various physiological stresses during dissemination. Exemplary, non-limiting NKCC inhibitors include bumetanide, ouabain furosemide, azosemide, torasemide, ethacrynic acid, dimethylamiloride, VU0463271, combinations thereof, and the like.

In some embodiments, the ion homeostasis modulator is a copper chelator. Elevated levels of copper are commonly observed in various cancer types, and its dysregulation has been linked to cancer cell growth, survival, and metastatic spread. Copper is involved in mitochondrial oxidative phosphorylation and mitochondrial copper depletion suppresses triple-negative breast cancer in mouse models. Exemplary copper chelators include, but are not limited to, tetrathiomolybdate (e.g., ammonium tetrathiomolybdate), penicillamine, trientine, ATN-224, clioquinol, pyrithione, combinations thereof, and the like.

In some embodiments, the ion homeostasis modulator is a calcium channel blocker. In metastatic cancer, calcium signaling facilitates various steps involved in the spread of cancer cells from the primary tumor to distant organs. Dysregulation of calcium homeostasis is commonly observed in cancer cells, and this disruption is closely linked to the aggressive behavior of metastatic tumors. Exemplary, non-limiting calcium channel blockers include, but are not limited to amlodipine, nifedipine, felodipine, isradipine, nicardipine, lercanidipine, nimodipine, verapamil, diltiazem, combinations thereof, and the like.

In some embodiments, treating the cancer metastasis further includes administering a modulator of extracellular matrix (ECM) interactions, optionally a therapeutic agent designed to silence ECM interactions. It will be appreciated that the silencing of ECM interactions-without added survival signals—may increase the susceptibility of healthy cells to anoikis. In some embodiments, the modulator of extracellular matrix (ECM) interactions is administered to the subject after the initial combination therapy. Without being bound by theory, the combination therapy may pharmacologically inactivate the anti-anoikis survival mechanisms, thereby allowing further targeting of the metastatic cancer, without as much detriment to healthy cells.

Therapeutic agents that target ECM interactions may act by blocking integrin signaling, degrading or inhibiting ECM components like collagen, hyaluronan, and laminin, and/or modulating enzymes involved in ECM turnover. Exemplary, non-limiting, ECM modulators include integrin inhibitors (e.g., cilengitide, talc, RGD peptide), collagen inhibitors (e.g., alantolacton, procollagen prolyl 4-hydroxylase inhibitor), laminin inhibitors (e.g., leptin) heparanase inhibitors (e.g., roneparstat, PG545), hyaluronidase inhibitors (e.g., PEGPH20), focal adhesion kinase inhibitors (e.g., defactinib, VS-4718), TGF-β inhibitors (e.g., Ly2157299, trabedersen), small molecule ECM disrupters (NSC 663284), combinations thereof, and the like.

### Pharmaceutical Compositions

Also provided herein are pharmaceutical compositions for treating cancer metastases, comprising a vascularization and/or tissue remodeling inhibitor, a suppressor of oxidative metabolism, an ion homeostasis modulator, and at least one pharmaceutically-acceptable excipient. Optionally, the composition may further comprise a modulator of extracellular matrix interactions. Any of the therapeutic agents disclosed herein are suitable for use in the pharmaceutical compositions.

The compositions may be prepared by any methods well known in the art of pharmacy, for example, using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carrier(s) for a given pharmaceutical composition and route(s) of administration.

As will be understood by those of skill in this art, the specific dose level of any agent for any particular subject will depend on a variety of factors, including the activity of the agent employed; the age, body weight, general health, and sex of the individual being treated; the time and route of administration; the rate of excretion; and the like. It is within the purview of the ordinary skilled artisan to determine the dose of the administered agent(s) for a particular subject.

Exemplary, non-limiting dosage amounts of representative therapeutic agents may be as follows: pazopanib: 200-800 mg/day; marimastat: 5-75 mg 2× daily or 10-50 mg daily; atovaquone: 250-750 mg 2× daily; DMSO: 0.5-1 g/kg bodyweight; tetrathiomolybdate: 10-40 mg 3× daily, optionally with an additional 10-60 mg at bedtime; bumetanide: 0.5-2 mg at 4-5 h intervals, up to 10 mg/day. In aspects, the dose of one or more therapeutic agents included in the combination therapy may be lower than the typical monotherapy dose for the agent(s).

The pharmaceutical compositions disclosed herein may be formulated for administration via a variety of routes. For example, pharmaceutical compositions may be formulated for enteral (e.g., oral, sublingual, buccal, or rectal), parenteral (e.g., intravenous, intramuscular, subcutaneous, intraarterial, intrathecal, intratumoral, etc.), intranasal, inhaled, vaginal, or transdermal administration. In some aspects, the pharmaceutical compositions are formulated for oral, intravenous, or intratumoral administration.

In aspects, pazopanib, marimastat, atovaquone, tetrathiomolybdate, DMSO, and bumetanide may be administered orally. In other aspects, DMSO may be administered topically. In other aspects, pazopanib, marimastat, atovaquone, tetrathiomolybdate, DMSO, and bumetanide may be administered intravenously or intratumorally by injection. In aspects, one or more agent(s) may be administered orally, while one or more other agent(s) may be administered intravenously or intratumorally by injection, or topically.

### EXAMPLES

The following examples are given by way of illustration are not intended to limit the scope of the disclosure.

### Example 1. Materials and Methods

### Drug Selection Criteria

First, drug candidates were tested individually for killing or cell-cycle-arresting adhesion-deprived cells (which represent good models for the cells that have been released from a primary tumor). All agents to be scrutinized have no effect at very low doses; at very high doses, they kill adherent and deadherent cells alike. These drugs would be seen as suitable candidates for further development if they suppressed adhesion-deprived cells in intermediate, pharmacologic concentration ranges without affecting the same cells in the adherent state (a drug that equally kills adherent and deadherent cells will likely also harm adherent non-transformed cells; such an agent would not be considered for further development). The initial assessments were performed on cells plated in conventional plastic dishes or on the polymer poly-HEMA, which prevents adhesion. Second, colony formation in soft agar was assessed, which depends on a combination of features, including the growth rate of the cells, the expression of gene products for invasiveness, and anti-anoikis. The ability of cells to form anchorage-independent colonies in soft agar reflects an important characteristic of transformation and correlates well with their ability to grow invasively in vivo. Results demonstrated 18 that benign tumor cells do not form colonies in this assay. By contrast, invasive cancer cells form soft agar colonies under the support from metastasis genes, such as Osteopontin. Third, true anti-metastasis activity was tested in murine models.

### Cells and Drugs

MIA PaCa-2 cells represent a hypertriploid pancreas carcinoma from a Caucasian male. PANC-1 are hypertriploid pancreatic epitheloid ductal carcinoma cells from a Caucasian male. MIA PaCa-2 and PANC-1 cells were cultured in DMEM 10% FBS (37° C., 5% CO₂). AsPC-1 is a metastasis-derived pancreatic adenocarcinoma from a Caucasian female (RPMI 10% FBS). MDA-MB-231 cells are triple negative, aneuploid breast adenocarcinoma cells. They were cultured in DMEM/F12 with 5% FBS. SW1116 (ATCC CCL-233, DMEM/F12 with 5% FBS) is a colorectal adenocarcinoma. RD are rhabdomyosarcoma cells (ATCC CCL-136, DMEM with 10% FBS).

Bumetanide, amlodipine, pazopanib, and marimastat were obtained from Selleck Chemicals (Houston, TX). Ammonium tetrathiomolybdate, verapamil, and N-acetyl cysteine (NAC) were obtained from Sigma-Aldrich (St. Louis, MO). DMSO and taurine were obtained from Fisher Scientific. Atovaquone was obtained from MyBiosource (San Diego, CA).

### Deadherent Survival

Cells were seeded at a density of 104 cells/well in 96-well plates, either uncoated or in poly(2-hydroxyethyl methacrylate) (poly-HEMA) coated wells (3 mg of poly-HEMA in 50 μL 95% ethanol, let dry overnight, then washed twice with PBS, followed by one wash with medium before initiation of the experiment). Drugs were added in triplicates at the indicated concentrations for an incubation time of 48 h. At the 48-h time point, the indicator WST-1 was added to each well at 10% v/v final concentration. At the 60-, 80-, and 120-minute time points, the absorbance was measured at 450 nm using a MRX-TC Revelation plate reader.

### Soft Agar Colony Formation

The capability of cells to grow under anchorage-independent conditions is a hallmark of transformation and correlates with tumor progression in vivo. The assay was performed in a 60-mm dish with a bottom layer of 2 mL 0.5% agar (Noble Agar, Sigma) in the culture medium that is designated to the cells. The bottom agar was allowed to be solidified in the biosafety cabinet. Cells were suspended in 0.2% agar for PANC-1 cells, MIA PaCa-2 cells, and B16-F10 cells, but in 0.15% agar for MDA-MB-231 cells and 4T1 cells and then plated (1×10⁵ cells/2 mL/dish) on top of the bottom layer. 400 μL culture medium with or without the indicated doses of drug treatment was added to cover the top agar layer every other day over 2 weeks for PANC-1, MIA PaCa-2 cells and over 3 weeks for MDA-MB-231 cells, respectively. The colonies in five microscopic fields (top, bottom, left, right and center) of each dish were photographed. Colony sizes were measured with Image-J. The colony frequency was found not to be altered, therefore we did not include it in the assessments. Each treatment was repeated thrice.

### Confocal Microscopy and Mitochondrial Size Assessment

Cells were maintained in soft agar for 5 days. Then, prewarmed (37° C.) staining solution containing 100 nM MitoTracker probe (Thermo Fisher Scientific) was added to the dish, followed by incubation for 30 min. The top agar was removed and placed into Nunc Glass Base dishes for visualization under a fluorescence microscope (Leica Stellaris 8 Confocal). The fluorescent areas were photographed and then quantified with Image-J.

### Confocal Microscopy and Assessment of Reactive Oxygen Species

The redox indicator dye di(acetoxymethyl ester) (6-carboxy-2′,7′-dichlorodihydrofluorescein diacetate) (DCFH-DA) was obtained from Thermo Fisher Scientific. Cells were maintained in soft agar for 16 h. At that time, they were incubated with 5 μM DCFH-DA for 30 min. For analysis, the top agar was separated from the bottom agar, before the cellular fluorescence intensity was visualized with confocal microscopy, photographed, and then measured in Image-J.

### In Vivo Analysis

Balb/c mice were injected intravenously with 0.2×10⁶ 4T1 cells. They were divided into four groups of four to five animals/group, encompassing no treatment (sham injections with PBS), combination chemotherapy (pazopanib+marimastat+DMSO+tetrathiomolybdate+bumetanide), subset treatment 1 (pazopanib+marimastat+DMSO), and subset treatment 2 (tetrathiomolybdate+bumetanide). The dosing was pazopanib 8 mg/kg, marimastat 15 mg/kg, tetrathiomolybdate 5 mg/kg, bumetanide 2 mg/kg, DMSO 200 μL/kg (all in a volume of 150-200 μL, the mice weighed close to 20 g). Treatment was administered every other day, starting either on Day 0 (metastasis prevention) or on Day 5 (treatment of established metastases) up to termination of the experiment at 14 days.

C57Bl/6 mice were injected intravenously (tail vein) or subcutaneously (flank) with 0.2×10⁶ B16-F10 cells. For the subcutaneous injections, care was taken not to damage the peritoneal lining. The bevel of the needle pointed outward, and the injection angle was as flat as feasible. They were divided into six groups of four animals/group, entailing no treatment (sham injections with PBS), combination chemotherapy (pazopanib+marimastat+DMSO+tetrathiomolybdate+bumetanide), subset treatment 1 leaving out pazopanib, subset treatment 2 leaving out marimastat, subset treatment 3 leaving out tetrathiomolybdate, and subset treatment 4 leaving out bumetanide. The dosing was pazopanib 8 mg/kg, marimastat 15 mg/kg, tetrathiomolybdate 5 mg/kg, bumetanide 2 mg/kg, DMSO 200 μL/kg (in a total volume of 150-200 μL). Delayed treatment added atovaquone at 30 mg/kg. Treatment was administered every other day, either starting on Day 0 (metastasis prevention) or on Day 5 (treatment of established metastases) up to termination of the experiment at 21 days.

At the termination date, the mice were sacrificed and tissue samples of tumor (after s.c. injection) and lungs (after i.v. injection) were fixed in formalin. Metastasis to the lungs was evaluated qualitatively by photographs and quantitatively by determining metastases counts and lung weights. Abdominal metastases were photographed (where feasible a small portion was saved in RNALater for RNASeq analysis), excised after formalin fixation and weighed for the quantification of tumor burden.

### Meta-Analysis

NCBI GEO datasets pertaining to cancer metastasis were analyzed. Following differential expression analysis of the array data, the identified genes were used as input for the pathway enrichment analysis, which identifies biological pathways (denoted as Gene Ontology terms or GO categories) that are associated with the upregulated or the downregulated genes. An FDR cut-off of 0.05 was used for selecting significant pathways. Enriched categories (GO terms) were evaluated. The p-value of a GO term was produced using Fisher's exact test (a hypergeometric distribution-based test), which is very sensitive to the numbers of genes in the up- or down-regulated gene list. From the tables, those categories affecting ion homeostasis according to UniProt were extracted.

### Statistics

The Wilcoxon-Wilcox test was used to assess significance in the WST-1 assays (with corroboration from the Student t-test where indicated) and the Student t-test was used for paired samples in soft agar and in vivo. ANOVA was applied where indicated. Significance was accepted at the p<0.05 level.

### Example 2. Anti-Metastasis Effect In Vivo

Example 2 details the treatment success against in vivo metastases with the drug combination of remodeling inhibitors/antioxidants/ion homeostasis regulators described herein. Balb/c mice, injected into the tail veins with 0.2×10⁶ 4T1 breast cancer cells, develop abundant foci of lung metastases after 14 days. The dosing was pazopanib 8 mg/kg, marimastat 15 mg/kg, tetrathiomolybdate 5 mg/kg, bumetanide 2 mg/kg, DMSO 200 μL/kg (in a volume of 200 μL)

Balb/c mice were injected intravenously (tail vein) with 0.2×10⁶ 4T1 cells. Four groups of four animals/group entailed no treatment (sham injections with PBS), combination chemotherapy (pazopanib+marimastat+DMSO+tetrathiomolybdate+bumetanide), subset treatment 1 (pazopanib+marimastat+DMSO), and subset treatment 2 (tetrathiomolybdate+bumetanide). Treatment was administered every other day, starting on Day 0 up to termination of the experiment at 14 days.

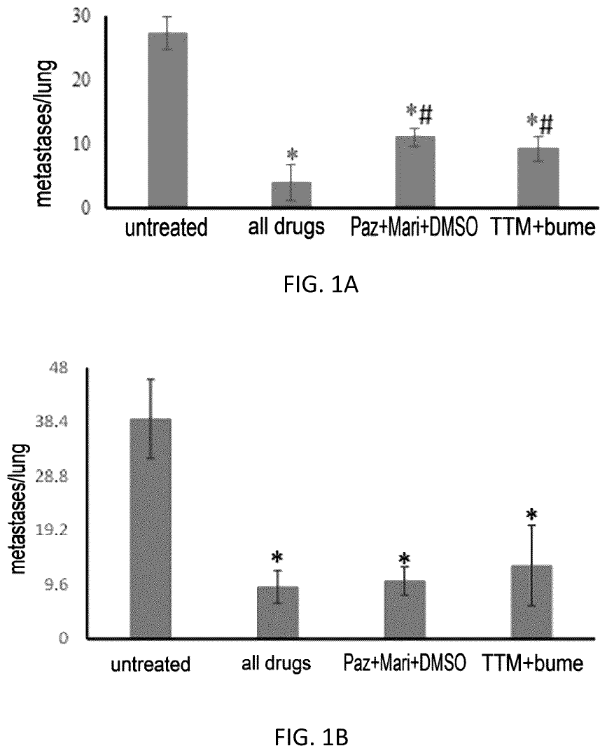

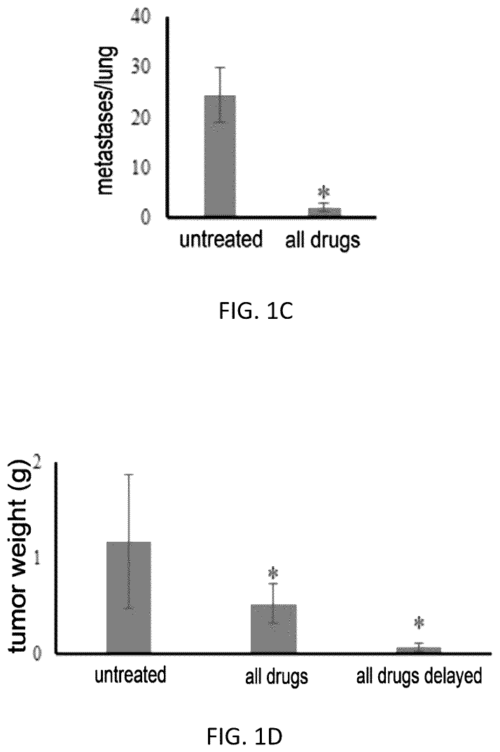

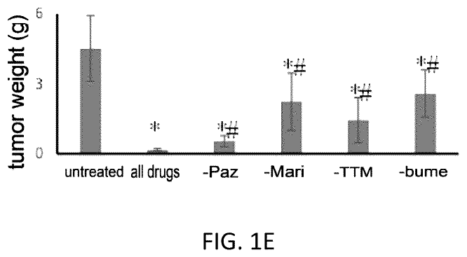

For treatment initiation from the day of injection, the combination of pazopanib+marimastat+DMSO+tetrathiomolybdate+bumetanide achieved 85% suppression of the metastatic burden, with the lungs of two mice being entirely free of macroscopically visible metastases. The treatments with subsets of the full combination therapy (pazopanib+marimastat+DMSO and tetrathiomolybdate+bumetanide) were partially efficacious at 60%-66% inhibition, as depicted in FIG. 1A.

In the clinical setting, cancers are often detected only after metastases have formed. Therefore, testing experimental anti-metastasis treatment for efficacy consecutive to a lag from the injection of tumor cells was also tested. Balb/c mice were injected intravenously (tail vein) with 0.2×10⁶ 4T1 cells. Four groups of four to five animals/group entailed no treatment (untreated: sham injections with PBS), combination chemotherapy (all drugs: pazopanib+marimastat+DMSO+tetrathiomolybdate+bumetanide), subset treatment 1 (pazopanib+marimastat+DMSO), and subset treatment 2 (tetrathiomolybdate+bumetanide).

Treatment was administered every other day, starting on Day 5 up to termination of the experiment at 14 days. Initiating treatment on day 5 after the intravenous injection of 0.2×10⁶ 4T1 cells and continuing every other day until termination on Day 14 still displayed high efficacy in suppressing the number of disseminated foci on the lungs (FIG. 1B).

C57Bl/6 mice were injected intravenously in the tail vein with 0.2×10⁶ B16-F10 cells. Two groups of five animals/group entailed no treatment (untreated: sham injections with PBS) and combination chemotherapy (all drugs: pazopanib+marimastat+DMSO+tetrathiomolybdate+bumetanide). Treatment was administered every other day, starting on Day 0 through the duration of the experiment for 21 days. Intravenous injection of 0.2×10⁶ B16-F10 cells led to abundant lung colonization that was suppressed with the drug combination (FIG. 1C). Twenty-one days after subcutaneous injection in the left flank with 0.2×10⁶ B16-F10 cells, untreated mice (sham injections with PBS) displayed copious abdominal metastases.

C57Bl/6 mice were injected subcutaneously in the left flank with 0.2×10⁶ B16-F10 cells. Two groups of five animals/group were injected with combination chemotherapy (pazopanib+marimastat+DMSO+tetrathiomolybdate+bumetanide) from Day 0 (all drugs) and Day 5 (all drugs delayed), one group of five mice entailed no treatment (untreated: sham injections with PBS). Under treatment with the combination therapy (pazopanib+marimastat+DMSO+tetrathiomolybdate+bumetanide) from Day 0 (full treatment) or from Day 5 (delayed full treatment), the intraperitoneal dissemination was substantially abrogated compared to the extensive tumor spread in the untreated group (FIG. 1D). Treatment with the full combination chemotherapy (pazopanib+marimastat+DMSO+tetrathiomolybdate+bumetanide) completely protected the treated mice from macroscopically visible metastases.

C57Bl/6 mice were injected subcutaneously in the left flank with 0.2×10⁶ B16-F10 cells. Six groups of four animals/group entailed no treatment (sham injections with PBS), combination chemotherapy (pazopanib+marimastat+DMSO+tetrathiomolybdate+bumetanide), subset treatment 1 leaving out pazopanib, subset treatment 2 leaving out marimastat, subset treatment 3 leaving out tetrathiomolybdate, and subset treatment 4 leaving out bumetanide (Paz, pazopanib; Mari, marimastat; TTM, ammonium tetrathiomolybdate; bume, bumetanide). The dosing was pazopanib 8 mg/kg, marimastat 15 mg/kg, tetrathiomolybdate 5 mg/kg, bumetanide 2 mg/kg, DMSO 4 μL (in a volume of 200 μL). Treatment was administered every other day, starting on Day 0 up to termination of the experiment at 21 days. The graph displays the total tumor burden of intraperitoneal mass plus primary tumor. Subset treatment 1 (leaving out pazopanib and treating with marimastat+DMSO+tetrathiomolybdate+bumetanide), subset treatment 2 (leaving out marimastat and treating with the combination of pazopanib+DMSO+tetrathiomolybdate+bumetanide), subset treatment 3 (leaving out tetrathiomolybdate and treating with pazopanib+marimastat+DMSO+bumetanide), and subset treatment 4 (leaving out bumetanide and treating with pazopanib+marimastat+DMSO+tetrathiomolybdate) all had partial effects as shown in FIG. 1E.

In sum, the drug combination proved highly efficacious with immediate and delayed treatment in two mouse models of cancer metastases.

### Example 3. Tissue Remodeling and Angiogenesis

Regarding vascularization and tissue remodeling, VEGF signaling is not limited to the blood vessels, but VEGF receptors are also expressed on cancer cells, and VEGF signaling occurs. Western blot analysis confirmed VEGFR2 expression in the MDA-MB-231, PANC-1, and MIA PaCa2 cell lines. This expression seemed to be constitutive as it was retained during growth under deadherent conditions. Thus VEGFR2 was identified as a viable drug target.

### Pazopanib Inhibits Soft Agar Colony Formation by Breast and Pancreatic Cancer Cells

Soft agar assays were performed to investigate whether pazopanib affects the anchorage-independent growth by MDA-MB-231 cells and PANC-1 cells. The drug inhibited colony formation in a dose-dependent manner in both cell lines, reaching significance at 0.5 μM (0.24 μg/mL) in MDA-MB-231 cells and at 0.25 μM (0.12 μg/mL) in PANC-1 cells. It inhibited soft agar colony formation by MIA PaCa-2 cells at 2.1 μM (1 μg/mL)

Pazopanib Suppresses Growth in Deadherent Breast and Pancreatic Cancer Cells with Lesser Effects on Plated Cells

Pazopanib was tested for its growth inhibition of the MDA-MB-231 breast cancer cell line, the pancreatic cancer cell line PANC-1, two additional human pancreatic cancer cell lines (AsPC1, MIA PaCa-2), and a human colon cancer cell line (SW1116) at 0-6 μg/mL for 48 hours under adherent or deadherent conditions.

In all cases, the deadherent cells were more sensitive to the drug effects than the adherent cells (data not shown). The agent significantly inhibited the growth of MDA-MB-231 cells on poly-HEMA at 0.4-2.0 μg/mL without compromising the same cells in their adherent state. At higher concentrations, both adherent and non-adherent cells had their WST-1 uptake suppressed. Pazopanib significantly decreased the cell growth of deadherent PANC-1 cells at a starting concentration of 0.2 μg/mL, while it increased adherent cell growth at 0.4-2 μg/mL. Above 2 μg/mL, the drug inhibited the growth of PANC-1 cells under both conditions.

### Example 4. Oxidative Metabolism

Deadherent survival and growth require induction of the oxidative metabolism. This may be due to an increased need for ATP once contact to the substratum and to other cells is lost. Distinct from the Warburg effect in primary tumors, increased mitochondrial respiration is needed.

Dimethyl Sulfoxide (DMSO) Inhibits the Colony Formation of Pancreatic Cancer Cells In Vitro but does not Impact the Growth of Deadherent or Plated Cancer Cells

To investigate whether peroxides react chemically to yield hydroxyl radicals, the hydroxyl radical scavenger DMSO was titrated into deadhesion and soft agar colony formation assays by MIA PaCa-2 or PANC-1 cells. DMSO was added at concentrations of 0.125%, 0.25%, 0.5%, 0.75% and 1% (v/v) every second day with the media replenishment. In the concentration range of 0.125%-0.25% (v/v), DMSO dose-dependently suppressed soft agar colony size, without impacting colony frequency.

In the same concentration range that completely suppressed soft agar colony formation, DMSO had no effect on the growth of PANC-1, MIA PaCa-2 or MDA-MB-231 cells when plated on plastic or on poly-HEMA.

### Atovaquone as an Anti-Metastasis Agent

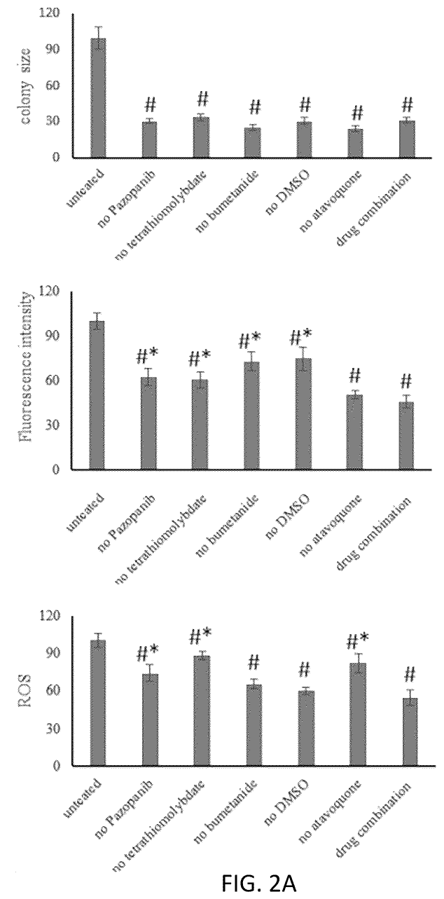

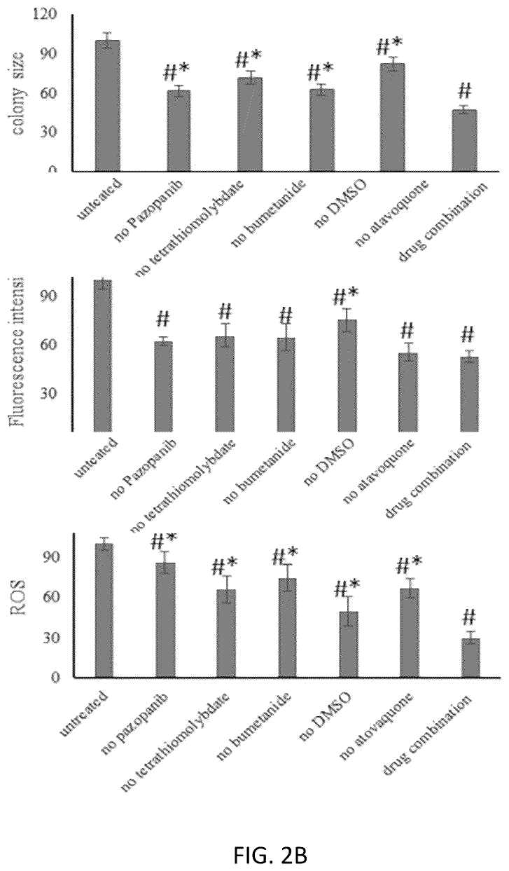

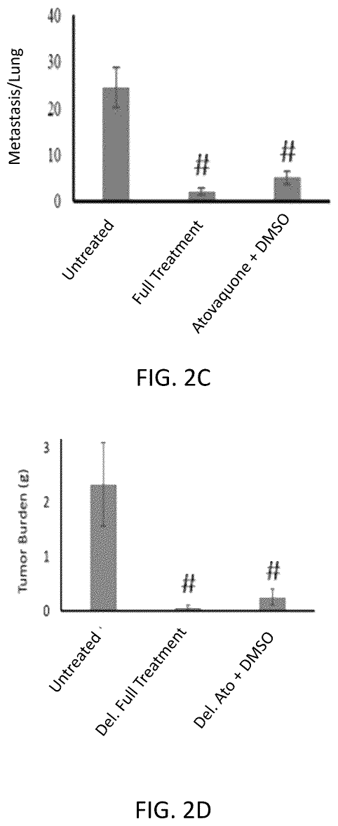

Atovaquone is an oral medication that inhibits oxidative phosphorylation. To test the efficacy of atovaquone, B16-F10 cells (FIG. 2A) or PANC-1 cells (FIG. 2B) were plated in soft agar. They were treated every other day with medium alone or with the indicated combinations of pazopanib (0.2 μg/mL), ammonium tetrathiomolybdate (0.25 μg/mL), bumetanide (7.5 μg/mL), dimethyl sulfoxide (DMSO, 0.125%, v/v), atovaquone (5 μg/mL). Colony size was measured after 11 days, mitochondrial size (according to a fluorescent dye) after 5 days, and intracellular peroxides (fluorescent indicator) after 16 h. The values were normalized to untreated=100%. Atovaquone suppressed the soft agar colony formation of B16-F10 cells and PANC-1 cells in a dose-dependent manner. This suppression was correlated to a reduction in intracellular reactive oxygen species according to fluorescence of the redox indicator DCFH-DA.

Atovaquone was also tested in vivo. C57Bl/6 mice were injected with B16-F10 cells. After 5 days, they were either untreated or treated every second day with the complete drug combination (Full Treatment, pazopanib+ammonium tetrathiomolybdate+bumetanide+DMSO+atovaquone) or DMSO (200 μL/kg) and atovaquone (30 mg/kg).

FIG. 2C depicts lung dissemination after intravenous injection, showing the metastasis count in the lungs after 21 days with or without treatment with atovaquone and DMSO. C57Bl/6 mice were injected intravenously with 2×10⁵ B16-F10 cells.

FIG. 2D graphically depicts peritoneal dissemination after subcutaneous injection. C57Bl/6 mice were injected subcutaneously in the left flank with 2×10⁵ B16-F10 cells. The graph shows the intraperitoneal tumor burden (g) of untreated mice compared to mice treated with the full combination and treated with atovaquone and DMSO (the primary tumors ranged from 0.1 to 0.51 g without differences among the treatment groups).

### Example 5. Ion Homeostasis

Ion homeostasis is substantially altered in cancer cells and even more so in metastasis. The effects of cognate antagonists on conventionally plated, adherent cancer cells versus the same cells under deadherent conditions were evaluated. The inhibition of soft agar colony formation was also evaluated.

### Bumetanide Inhibits the Colony Formation of Pancreatic Cancer Cells in Soft Agar and Inhibits Cell Proliferation of Deadherent Breast Cancer Cells.

Bumetanide is a specific inhibitor of Na—K-2Cl cotransport. The effects of bumetanide on anchorage independence (cell proliferation and soft agar colony formation) were evaluated (data not shown). The agent significantly decreased the colony formation of MDA-MB-231 cells by 56%-82% at a concentration range of 7.5-30 μg/mL. It also suppressed the colony formation of MIA PaCa-2 cells by 31%-52% at 7.5-30 g/mL.

The growth of MDA-MB-231 cells on poly-HEMA was inhibited in a dose-dependent manner by bumetanide treatment, with significant inhibition at 15-35 μg/mL, without affecting plated cells. The drug suppressed both adherent and deadherent cells above 50 μg/mL. In a concentration range of 1-5 μg/mL, bumetanide suppressed the proliferation of MIA PaCa-2 cells on poly-HEMA.

### Ammonium Tetrathiomolybdate Inhibits the Colony Formation of Cancer Cells in Soft Agar

The effect of ammonium tetrathiomolybdate on anchorage independence (cell proliferation and soft agar colony formation) was investigated in MDA-MB-231 cells, PANC1 cells, and MIA PaCa-2 cells. The drug inhibited the colony size of MDA-MB-231 cells by 43%, 75%, 93%, and 94% at 0.5, 1, 2, and 4 μg/mL, respectively. It similarly inhibited the colony formation of PANC-1 cells in a dose-dependent manner, 30%-90% at 0.25-4 μg/mL. Soft agar colony formation by MIA PaCa-2 cells was suppressed by 33%-54% by 0.25-2 μg/mL ammonium tetrathiomolybdate.

### Example 6. Drug Combinations

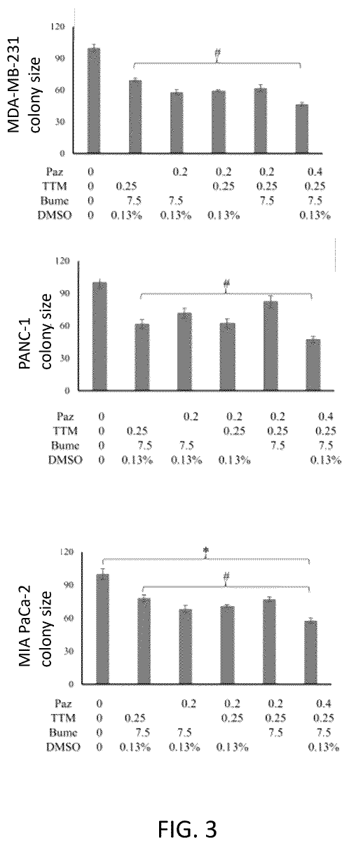

The drug combination of pazopanib (Paz), ammonium tetrathiomolybdate (TTM), bumetanide (Bume), and dimethyl sulfoxide (DMSO) was tested for its inhibitory effect on soft agar colony formation by MDA-MB-231 cells (top panel), PANC-1 cells (middle panel), and Mia PaCa-2 cells (bottom panel), as shown in FIG. 3. To assess the contributions by the individual drugs, the loss of inhibition in soft agar colony formation was tested by removing one agent at a time.

The chosen concentrations represent the 25% percent inhibitory dose individually. The left bar indicates untreated cells, the right bar displays the result in the presence of the full drug cocktail. The bars in between are reflective of the colony sizes when one drug was removed at a time. The error bars are sem. The values were normalized to untreated=100%. * indicates that all treatments are significantly different from untreated (left bar) at p<0.05. # indicates that all partial treatments were significantly different from the complete combination therapy (right bar) at p<0.05 (ANOVA).

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method of treating cancer metastasis in a subject in need thereof, the method comprising administering to the subject a combination therapy comprising:
(a) a vascularization and/or tissue remodeling inhibitor;
(b) a suppressor of oxidative metabolism; and
(c) an ion homeostasis modulator.

2. The method according to claim 1, wherein the vascularization and/or tissue remodeling inhibitor is a broad spectrum vascular endothelial growth factor receptor (VEGFR) inhibitor or a matrix metalloproteinase (MMP) inhibitor.

3. The method according to claim 2, wherein the broad spectrum VEGFR inhibitor is selected from the group consisting of pazopanib, bevacizumab, sunitinib, cabozantinib, sorafenib, regorafenib, lenvatinib, nintedanib, apatinib, axitinib, vandetanib, tivozanib, ramucirumab, fruquintinib, ponatinib, and combinations thereof.

4. The method according to claim 3, wherein the broad spectrum VEGFR inhibitor is pazopanib.

5. The method according to claim 2, wherein the MMP inhibitor is selected from the group consisting of marimastat, ilomastat, batimastat, MMI-270, MMI-166, prinomastat, ABT-770, cipemastat, RS-130830, CAS Reg. No. 239796-97-5, rebimastat, tanomastat, Ro 28-2653, and combinations thereof.

6. The method according to claim 5, wherein the MMP inhibitor is marimastat.

7. The method according to claim 1, wherein the suppressor of oxidative metabolism is selected from the group consisting of dimethyl sulfoxide (DMSO), atovaquone, N-acetylcysteine (NAC), taurine, and combinations thereof.

8. The method according to claim 7, wherein the suppressor of oxidative metabolism is DMSO or atovaquone.

9. The method according to claim 1, wherein the ion homeostasis modulator is an NKCC inhibitor or a copper chelator.

10. The method according to claim 1, wherein the ion homeostasis modulator is selected from the group consisting of bumetanide, azosemide, ouabain furosemide, torasemide, ethacrynic acid, dimethylamiloride, VU0463271, tetrathiomolybdate, and combinations thereof.

11. The method according to claim 9, wherein the NKCC inhibitor is bumetanide.

12. The method according to claim 9, wherein the copper chelator is tetrathiomolybdate.

13. The method according to claim 1, further comprising a modulator of extracellular matrix interactions.

14. The method according to claim 1, wherein the cancer metastasis is selected from the group consisting of pancreatic cancer, breast cancer, colorectal cancer, and rhabdomyosarcoma.

15. The method according to claim 1, wherein the combination therapy is administered enterally or parenterally.

16. The method according to claim 15, wherein the parenteral administration comprises intravenous or intratumoral administration.

17. A pharmaceutical composition comprising:
(a) a vascularization and/or tissue remodeling inhibitor;
(b) a suppressor of oxidative metabolism;
(c) an ion homeostasis modulator; and
(d) at least one pharmaceutically-acceptable excipient.

18. The pharmaceutical composition according to claim 17, formulated for enteral or parenteral administration.

19. The pharmaceutical composition according to claim 17, wherein the vascularization and/or tissue remodeling inhibitor is selected from the group consisting of pazopanib, bevacizumab, sunitinib, cabozantinib, sorafenib, regorafenib, lenvatinib, nintedanib, apatinib, axitinib, vandetanib, tivozanib, ramucirumab, fruquintinib, ponatinib, marimastat, ilomastat, batimastat, MMI-270, MMI-166, prinomastat, ABT-770, cipemastat, RS-130830, CAS Reg. No. 239796-97-5, rebimastat, tanomastat, Ro 28-2653, and combinations thereof.

20. The pharmaceutical composition according to claim 17, wherein the suppressor of oxidative metabolism is selected from the group consisting of DMSO, atovaquone, NAC, taurine, and combinations thereof.

21. The pharmaceutical composition according to claim 17, wherein the ion homeostasis modulator is selected from the group consisting of bumetanide, tetrathiomolybdate, and combinations thereof.

22. The pharmaceutical composition according to claim 17, comprising:
pazopanib and/or marimastat;
DMSO and/or atovaquone; and
bumetanide and/or tetrathiomolybdate.
